(19) United States
(12) Patent Application Publication
(10) Pub. No.: US 2022/0084661 A1
(43) Pub. Date: Mar. 17, 2022

(54) Automated Mobile Pharmacy

(71) Applicant: KYLER B. FOLSE, RYANE, LA (US)

(72) Inventor: KYLER B. FOLSE, RYANE, LA (US)

(21) Appl. No.: 17/021,418

(22) Filed: Sep. 15, 2020

(51) Int. Cl. G16H 40/20; G16H 40/67; G06Q 50/28; G16H 20/10; G06Q 30/06; G06Q 30/00; G16H 10/60; G16H 50/20; G05B 19/4155; B60P 3/025

(52) U.S. Cl. CPC G16H 40/20; G16H 40/67; G06Q 50/28; G16H 20/10; G06Q 30/0645; H04N 7/181; G16H 10/60; G16H 50/20; G05B 19/4155; B60P 3/025; G05B 2219/40269; G06Q 30/0185

(57) ABSTRACT

An automated, mobile pharmacy system is disclosed. The invention is comprised of a small structure or conventional shipping container converted into a secure pharmacy with robotic systems and artificial intelligence software operations that can be delivered to communities around the world. Embodiments of the invention can be rented and managed by pharmacists. The system within the pharmacy conducts automated prescription filling and includes drive-through pickups at an attached kiosk, scheduled walk-in pickups at internal lockers, mail deliveries, and home delivery services. During drive through and internal locker pickups, users present their identification and security code to the window to pick up medications. During scheduled walk in pickups, users are notified when a prescription is ready and enter the pharmacy using a security code and retrieve their medications from secure lockers inside. An object of the invention is to enhance secure access to prescription medications.

### FIELD OF THE INVENTION

The present invention generally relates to pharmacies More specifically, it relates to an automated, mobile pharmacy system.

### BACKGROUND

The beginnings of pharmacy can be traced back thousands of years. In Greek legend, Asclepius, (the god of healing), delegated the duty of compounding his remedies to Hygieia. Hygieia was his apothecary or pharmacist. The physician-priests of Egypt were essentially divided into two classes: those who visited the sick and those who remained in the temple and prepared remedies for the patients. In ancient Greece and Rome and during the Middle Ages in Europe, the art of healing recognized a separation between the duties of the physician and those of the herbalist, who supplied the physician with the raw materials from which to make medicines. The Arabian influence in Europe during the 8th century AD, however, brought about the practice of separating the duties of the pharmacist and the physician. The trend toward specialization was later reinforced by a law enacted by the city council of Bruges in 1683, forbidding physicians to prepare medications for their patients. In America, Benjamin Franklin took a pivotal step in keeping the two professions separate when he appointed an apothecary to the Pennsylvania Hospital. The development of the pharmaceutical industry since World War II led to the discovery and use of new and effective drug substances. It also changed the role of the pharmacist. The scope for extemporaneous compounding of medicines was much diminished and with it the need for the manipulative skills that were previously applied by the pharmacist to the preparation of bougies, cachets, pills, plasters, and portions. The pharmacist continues, however, to play a role by providing advice and information; by formulating, storing, and providing correct dosage forms; and by assuring the efficacy and quality of the dispensed medicinal product. With the advent of the information age, the medical industry took advantage of these systems to streamline patient documentation and how care is offered to patients. Today patients can discuss symptoms and diagnoses with their health care providers remotely without ever stepping foot in an office. Unfortunately, most of the pharmaceutical industry has not fully-leveraged such technology and still requires patients to visit central locations to obtain their medications. United States Patent No. US20130030824A1 granted to Fayek Todary Michael disclosed a mobile pharmacy with a transport vehicle; however, it does not incorporate automated prescription filling. United States patent No. United States patent No. U.S. Pat. No. 7,848,934B2 granted to Paul Kobylevsky and Valery Gurovich disclosed an automated pharmacy; however, it is not mobile. What is needed is a mobile, automated pharmacy system to serve multiple communities.

### SUMMARY OF THE INVENTION

The device herein disclosed and described provides a solution to the shortcomings in the prior art through the disclosure of an automated, mobile pharmacy. An object of the invention is to provide a cost-effective means to allow pharmacies to exist in multiple locations within the community. Portable buildings and conventional shipping containers are converted into mobile pharmacies that can be transported using standard trailer trucks and dropped off at multiple locations.

Another object of the invention is to automate the prescription filling process using robotics. The doctor uploads an electronic prescription to the patient profile on the cloud network when they issue a new or reoccurring prescription. Robots collect medications from a secured repository inside the structure and dispense measured amounts into the appropriate vessels and bottles. All automated prescription activities are monitored for quality assurance and include various code scans during the process to rule out errors and maintain security.

Another object of the invention is to allow users to order medications remotely and then pick them up at their convenience—rather than waiting for a pharmacist to fill their order. For example, the doctor uploads an electronic prescription to the patient profile on the cloud network when they issue a new or reoccurring prescription. The patient is notified that the order is ready and asks them their pickup preference. The patient opts to pick up the prescription at the mobile pharmacy. After processing the order, the robotic system places the medications into a dedicated locker for the customer. When the customer is ready to take delivery, they visit the mobile pharmacy, provide proof of identification at a dedicated locker with scanning and retrieve their medications. The system also accepts patient co-pays at the kiosk.

Another object of the invention is to provide enhanced security measures for the mobile pharmacy. The portable building is outfitted with both external and internal closed-circuit monitoring at all times. During times of inactivity, motion detectors provide constant surveillance and if unauthorized activity is detected, the systems automatically notify managers and local authorities in real time.

Another object of the invention is to allow users to pick up medications without entering the mobile pharmacy. The doctor uploads an electronic prescription to the patient profile on the cloud network when they issue a new or reoccurring prescription. An automated drive-through window is positioned on one side of the portable building. After security measures are satisfied by means of the onboard kiosk, robotic arms deliver medications to customers through the drive through window. The system also accepts patient co-pays at the drive-through window.

Another object of the invention is to allow medications to be delivered by mail or courier to a user's residence. The doctor uploads an electronic prescription to the patient profile on the cloud network when they issue a new or reoccurring prescription. After a prescription is filled, robots store medications in dedicated, secure lockers that can be accessed by Postal Service agents and local, commercial couriers (after scanning their identities) who then deliver it to the customer at home.

Another object of the invention is to allow for enhanced security and quality control management through the use of artificial intelligence (AI). The AI in the mobile pharmacy is constantly learning about drug profiles, new medications, and drug interactions to make accurate predictions from data gathered around the world in an effort to better serve customers and make appropriate recommendations when needed.

Another object of the invention is to automate pharmaceuticals inventory management. For example, the computer systems that govern the mobile pharmacy and oversee robotic activity also monitor drug stores within the structure. The software routinely monitors physical parameters (such as temperature, humidity etc.) as well as expiration dates of drugs in order to prevent spoilage. Supplies are monitored in real time and restocking requests are performed automatically by sending electronic notifications to suppliers and pharmaceutical reps.

Another object in the invention is to allow pharmacists, and pharmacy companies to rent out and manage the mobile pharmacy in various locations of their choosing.

It is briefly noted that upon a reading this disclosure, those skilled in the art will recognize various means for carrying out these intended features of the invention. As such it is to be understood that other methods, applications and systems adapted to the task may be configured to carry out these features and are therefore considered to be within the scope and intent of the present invention, and are anticipated. With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention. As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. The objects features, and advantages of the present invention, as well as the advantages thereof over existing prior art, which will become apparent from the description to follow, are accomplished by the improvements described in this specification and hereinafter described in the following detailed description which fully discloses the invention, but should not be considered as placing limitations thereon.

### BRIEF DESCRIPTION OF THE FIGURES

The accompanying drawings, which are incorporated herein and form a part of the specification, illustrate some, but not the only or exclusive, examples of embodiments and/or features.

FIG. 1 shows a perspective view of the invention in use.

FIG. 2 shows a perspective view of the prescription filling automation.

FIG. 3 shows a close-up view of the robotic automation.

FIG. 4 shows a side view of the pickup kiosk.

FIG. 5 shows a side view of the pickup kiosk.

FIG. 6 shows a close-up view of the pickup kiosk.

FIG. 7 shows a representative view of the invention's process.

Other aspects of the present invention shall be more readily understood when considered in conjunction with the accompanying drawings, and the following detailed description, neither of which should be considered limiting.

### DETAILED DESCRIPTION OF FIGURES

In this description, the directional prepositions of up, upwardly, down, downwardly, front, back, top, upper, bottom, lower, left, right and other such terms refer to the device as it is oriented and appears in the drawings and are used for convenience only; they are not intended to be limiting or to imply that the device has to be used or positioned in any particular orientation. Conventional components of the invention are elements that are well-known in the prior art and will not be discussed in detail for this disclosure.

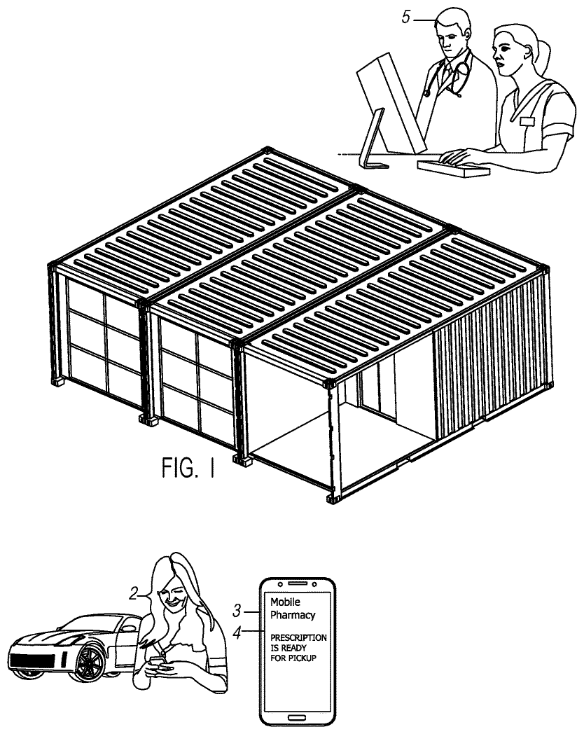

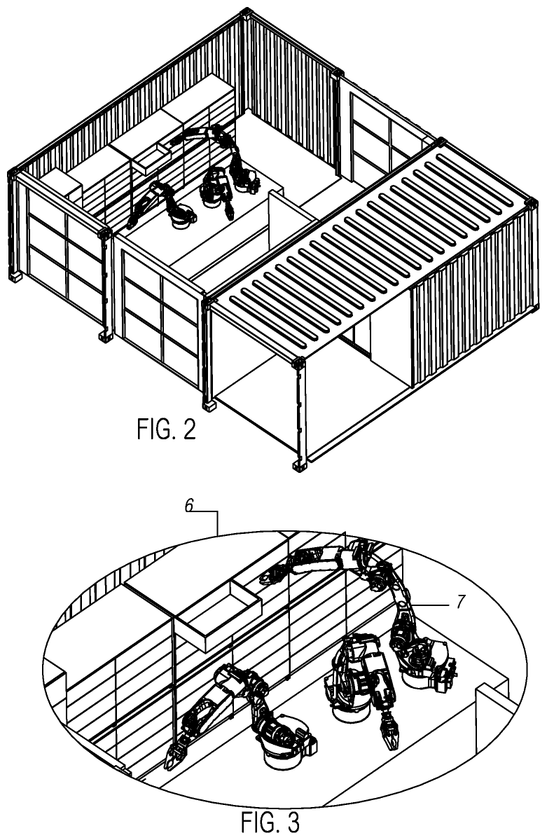

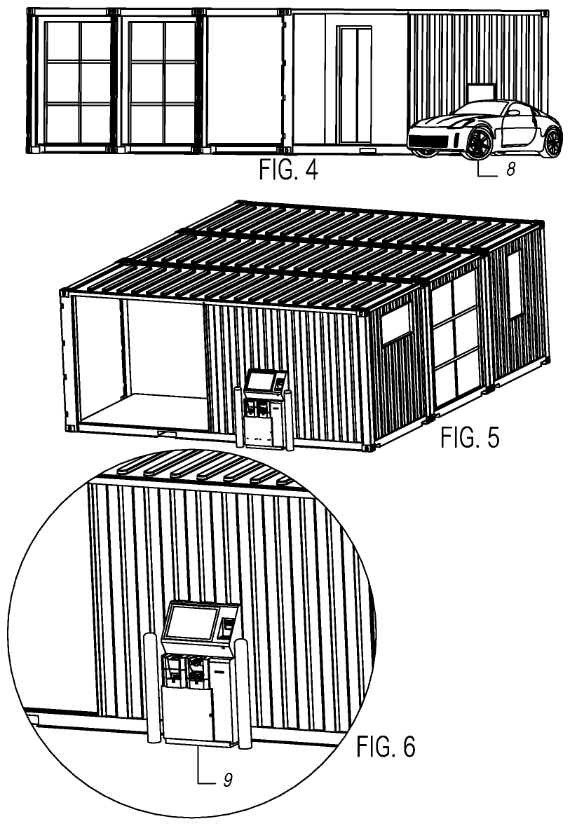

FIG. 1 shows a perspective view of the preferred embodiment of the invention 1 comprised of a plurality of portable buildings including but not limited to conventional shipping containers etc. having a multitude of secure windows, doors and prescription storage lockers etc. The structure having a multitude of internal and external security cameras. The figure showing patient 2 logging into the mobile pharmacy software 4 and receiving a message on smart phone 3. The figure also showing the mobile pharmacy being managed by pharmacist 5 remotely. FIGS. 2 and 3 showing automated robotic arms 7 retrieving medications from secure storage drawers 6 and preparing them for deliveries and pickups. Said automated robotic arms being controlled by workstations with software connected remotely to a cloud network that is governed by a pharmacist. FIGS. 5-6 showing another embodiment of the invention having a pickup kiosk 9 that allows users to verify their identity (by scanning barcodes, QR codes, facial recognition, entering passcodes on the touchscreen, etc.), make payments (using a credit card, cash, PayPal and Venmo etc.), and then receiving their medications through the kiosk.

Those of skill in the art will recognize that mobile applications are written in several languages include, by way of non-limiting examples, C, C++, C#, Objective-C, Java, Javascript, Pascal, Object Pascal, Python, Ruby, VB.NET, WML, and XHL/HL with or without CSS, or combinations thereof. The app in invention 1 is also compatible with a plurality of operating systems such as, but not limited to: Windows, Apple, and Android, and compatible with a multitude of hardware platforms such as, but not limited to: personal desktops, laptops, tablets, smartphones and the like. Suitable mobile application development environments are available from several sources. Commercially available development environments include, by way of non-limiting examples, AirplaySDK, alcheMo, Appcelerator, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android SDK, BlackBerry SDK, BREW SDK, Palm OS SDK, Symbian SDK, webOS SDK, and Windows Mobile SDK. Those of skill in the art will recognize that several commercial forums are available for distribution of mobile applications including, by way of non-limiting examples, Apple App Store, Google Play, Chrome Web Store, BlackBerry App World, App Store for Palm devices, App Catalog for webOS, Windows Marketplace for Mobile, Ovi Store for Nokia devices, Samsung Apps, and Nintendo DSi Shop.

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications. In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe Flash Player, Microsoft Silverlight, and Apple QuickTime.

In view of the disclosure provided herein, those of skill in the art will recognize that several plug-in frameworks are available that enable development of plug-ins in various programming languages, including, by way of non-limiting examples, C++, Delphi, Java™, PHP, Python, and VB .NET, or combinations thereof. Web browsers (also called Internet browsers) are software applications, designed for use with network-connected digital processing devices, for retrieving, presenting, and traversing information resources on the World Wide Web. Suitable web browsers include, by way of non-limiting examples, Microsoft Internet Explorer, Mozilla Firefox, Google Chrome, Apple Safari, Opera Software Opera, and KDE Konqueror. In some embodiments, the web browser is a mobile web browser. Mobile web browsers (also called micro-browsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google Android browser, RIM BlackBerry Browser, Apple Safari, Palm Blazer, Palm WebOS Browser, Mozilla Firefox for mobile, Microsoft Internet Explorer Mobile, Amazon Kindle Basic Web, Nokia Browser, Opera Software Opera Mobile, and Sony PSP™ browser. Software Modules.

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

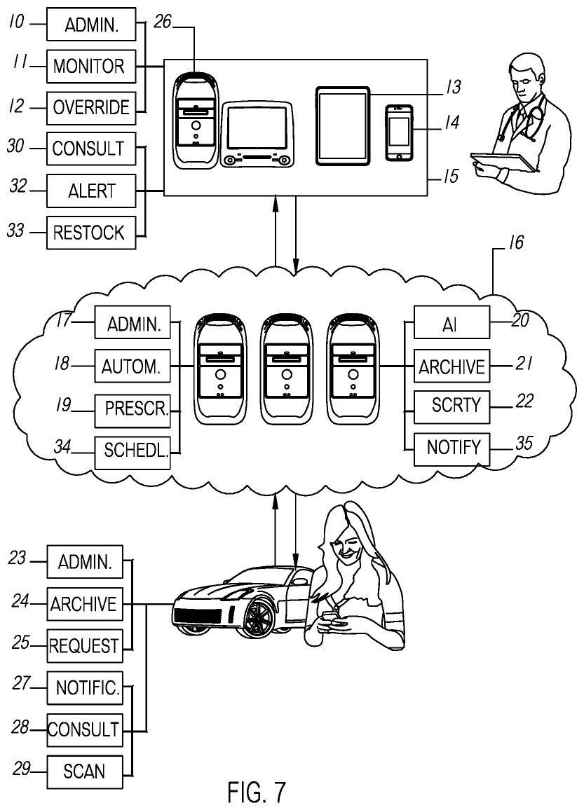

FIG. 7 showing a representative view of the preferred embodiment of the invention's process. Pharmacy managers having access to functions that include but are not limited to: administrative operations 10 (entering their demographics and credentials, logging into the invention website, purchasing a rental subscription to the mobile pharmacy, monitoring medicinal stores for expirations etc.); monitoring the mobile pharmacy 11 (scanning security cameras, reviewing monetary transactions and sales, etc.); overriding automated robotic systems 12 (emergency shutdowns, de-energizing robotic arms, controlling the kiosk etc.); providing real-time video consultation with patients remotely 30; receiving emergency alerts 32 (break-ins, tapering, fire emergencies etc.); and reviewing and restocking medical supplies 33 (physical access to the mobile pharmacy—including all doors and kiosk entries etc.). The figure also showing operations available to a patient, including but not limited to: administrative functions 23 (entering demographics, pharmaceutical needs, and purchase history etc.); medical archives 24 (insurance providers, prescription status, current medications, pickup and delivery history etc.); prescription requests 25 (status, delivery type, prescription filled dates, etc.); notifications 27 (prescription changes, prescription status, drug news via SMS, email, text etc.); remote consultations scheduled with pharmacist 28 (including AI algorithms that are constantly learning about drug profiles, new medications, and drug interactions to make accurate predictions from meta data gathered); and identity scan operations 29 (profile barcode, QR code, retinal scan, finger scan, facial recognition, kiosk scans etc.).

The pharmacist and the patient being connected to the mobile pharmacy by means of cloud network 16. Cloud network 16 having routines and operations such as but not limited to: administrative management 17 (managing stakeholder accounts, subscriptions, payments, rental operations etc.); automated prescription filling algorithms 18 (preprogrammed and dynamic robotic arm operations); prescription filling and verification systems 19 (retrieval routines and security recordings etc.); prescription pickup scheduling 34 (notifications, Google calendars, etc. or patient pickups, postal deliveries and courier visits etc.); artificial intelligence operations 20 (predicting proper medical filled processes and automated recommendations to patients, etc.); archive database 21 (patient and prescription history, digital security logs, medical supply orders, pharmacy transactions and history etc.); security surveillance and network encryptions 22; and notifications to stakeholders 35 (SMS, texts, emails etc.).

It is additionally noted and anticipated that although the device is shown in its most simple form, various components and aspects of the device may be differently shaped or slightly modified when forming the invention herein. As such those skilled in the art will appreciate the descriptions and depictions set forth in this disclosure or merely meant to portray examples of preferred modes within the overall scope and intent of the invention, and are not to be considered limiting in any manner. While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the scope of the invention.

What is claimed is:

1. A mobile pharmacy comprised of the following parts:
a) a portable building for storing and dispensing medications;
b) automated robotic arms for preparing medications for pickups and deliveries;
c) computer systems for remotely managing the mobile pharmacy;
d) a kiosk for customer pickups;
e) lockers for customer pickups and postal and courier delivery pickups.
f) a software application; and
g) a cloud network.

2. The mobile pharmacy of claim 1 wherein the mobile pharmacy can be rented by pharmacists and pharmacy companies.

3. The mobile pharmacy of claim 1 wherein the mobile pharmacy can transported to multiple locations to serve communities.

4. The mobile pharmacy of claim 1 wherein the portable building has exterior and interior security cameras and systems.

5. The mobile pharmacy of claim 1 wherein the computer systems and automated robotic arms can be monitored and shut down by the pharmacist remotely.

6. The mobile pharmacy of claim 1 wherein the kiosk and lockers have customer identification verification systems.

7. The mobile pharmacy of claim 1 wherein the software application manages patient profiles and pharmacist demographics, alerts stakeholders of prescription problems, and archives transactions and medication histories.

8. The mobile pharmacy of claim 1 wherein the software application allows doctors to add prescription refills to a patient's profile and automatically notifies the patient of the prescription status.

9. The mobile pharmacy of claim 1 wherein the software application allows patients to request prescription refills, schedule home deliveries, and postal deliveries of medications remotely online.

10. The mobile pharmacy of claim 1 wherein the software application allows pharmacists to monitor the mobile pharmacy security operations, prescription filling, drug expiration dates and restock supplies for the mobile pharmacy in real time online.

11. The mobile pharmacy of claim 1 wherein the software application provides consultations to patients by means of AI predictive algorithms.
